(19) United States
(12) Patent Application Publication
(10) Pub. No.: US 2004/0228898 A1
(43) Pub. Date: Nov. 18, 2004

(54) Thermopolymer composition and related methods

(76) Inventors: Anthony C. Ross, Hollywood, SC (US); Peter Guagliano, Brooklyn, NY (US)

Correspondence Address: NU VASIVE, INC., 10065 OLD GROVE ROAD, SAN DIEGO, CA, 92131, US

(21) Appl. No.: 10/849,756

(22) Filed: May 20, 2004

Related U.S. Application Data
(63) Continuation of application No. PCT/US02/37541, filed on Nov. 21, 2002.

(51) Int. Cl. A61K 33/24

(52) U.S. Cl. 424/423; 424/617

(57) ABSTRACT

A thermopolymer composition is disclosed suitable for use in filling voids within a human body, including but not limited to orthopedic joints (i.e. the discs of the spine and joints of the extremities), spaces between bone fractures or separations, and/or voids created within muscle and/or viscera for the purpose of tissue augmentation. The thermopolymer composition of the present invention may be heated and injected into the body in flowable form and thereafter cooled to body temperature to become a flexible, yet relatively solid material.

### CROSS-REFERENCES TO RELATED APPLICATIONS

This application is a continuation under 35 U.S.C. 111(a) of PCT Patent Application Serial No. PCT/US02/37541, filed Nov. 21, 2002 and published on Jun. 5, 2003 as WO 03/045274 which is incorporated herein by reference.

### BACKGROUND OF THE INVENTION

I. Field of the Invention

This invention relates generally to a thermopolymer composition that may be used to fill voids within a human body, including but not limited to orthopedic joints (i.e. the discs of the spine and joints of the extremities), spaces between bone fractures or separations, and/or voids created within muscle and/or viscera for the purpose of tissue augmentation. More particularly, the thermopolymer composition of the present invention may be heated and injected into the body in flowable form and thereafter cooled to body temperature to become a flexible, yet relatively solid material.

II. Description of Related Art

Voids may occur in the body, either through natural causes, injury or medical procedures. As used herein, “void” means any space or gap existing between and/or within biologic structures within a body, including but not limited to structures forming part or a portion of orthopedic joints, bones, muscle and/or viscera. For example, excessive wear may cause a void in an orthopedic joint, a broken bone may result in gaps in the fracture site, arthoscopic surgery may require removing bone or cartilage, and tissue augmentation may require injecting a compound into muscle and/or viscera and thereby create a void. In these and other instances, it may be useful to fill the void with a resilient, non-dispersing material. In other applications, it is desirable to deliberately form a void, for example, between disks or within muscle and/or viscera, or to increase the volume of an existing void.

Whatever the cause of the void, it is desirable to fill the void with a composition that is physiologically acceptable to the human body, and which allows the area to retain normal function and characteristics. For example, proper joint function includes cushioning the forces on the joint and minimizing wear and abrasion to the joint. The material, when set, should therefore be resilient, pliable, and non-dispersing.

U.S. Pat. Nos. 6,183,518, 6,206,921, and 6,264,659 disclose processes for which the present invention may be useful. These patents describe a process for repairing intervertebral disks of mammals by removing nucleus pulposis and injecting a resilient, pliable, non-dispersing material in its place. The present invention may be used with the technology disclosed in these patents to provide an improved resilient, non-dispersing material for filling the void created by removal of the nucleus pulposis and surrounding tissues.

One component of a resilient, non-dispersing material may include an isoprene powder, such as gutta percha. Gutta percha and other isoprene materials have been used for example, in dental applications. U.S. Pat. No. 6,126,446 describes a composition comprising gutta percha and other isoprene powders for filling tooth root canals. U.S. Pat. No. 4,632,977 offers other filling compositions based on isoprene materials, such as gutta percha. Other patents of interest include U.S. Pat. No. 5,047,055, disclosing a prosthetic nucleus for a vertebral disc comprised of hydrogel; U.S. Pat. No. 5,545,229, disclosing a replacement disc using elastomeric material in its nucleus and annulus; and U.S. Pat. No. 5,800,549, disclosing a method and apparatus for injecting an elastic spinal implant into a cavity in a spinal disc so as to treat disc degeneration.

Current formulations of material injectable into the spine and other parts of the body have inherent limitations. For example, some materials may be inflammatory or are otherwise incompatible with joints of many patients. Other materials may also have limited strength and durability, and may decay or degrade with time.

The present invention is directed at addressing the need for an improved void-filling composition and eliminating, or at least reducing the effects of, the above-described problems with the prior art.

### SUMMARY OF THE INVENTION

The present invention addresses the above-identified need and overcomes the problems with the prior art by providing a thermopolymer composition and related methods for filling a void within a human body, wherein the thermopolymer composition has improved mechanical and chemical properties, making it stronger, more durable, and more compatible with the human body. The thermopolymer composition of the present invention is suitable for filling any number of voids (which, as used herein, is defined as any space or gap existing between and/or within biologic structures within a human body). These voids may be formed via natural causes, injury, and/or medical procedures and may, by way of example only, include spaces or gaps formed, created and/or otherwise existing within part or a portion of orthopedic joints (i.e. the discs of the spine and joints of the extremities), bones, muscle and/or viscera. Suitable applications for the thermopolymer composition of the present invention include, but are not limited to, disc nucleus replacement (following partial or full discectomy), vertebroplasty, and tissue augmentation procedures. Illustrative examples of tissue augmentation procedures may include any number of restorative and/or reconfiguration procedures, including but not limited to reconstructive facial surgery, breast augmentation, and urinary incontinence treatment (by injecting the thermopolymer composition of the present invention into the urinary sphincter to serve as a bulking agent).

According to one broad aspect of the present invention, the thermopolymer composition includes a thermopolymer matrix having a dispersion compound therein. The thermopolymer matrix may comprise any number of suitable thermopolymer materials capable of being heated and injected in a flowable or molten state into a body (either into an existing void or creating a void) and thereafter cooling to body temperature to become a flexible, yet relatively solid material. In a preferred embodiment, the thermopolymer matrix is gutta percha. In alternate embodiments, the thermopolymer matrix may comprise balata, polyisoprene and/or any mixture of gutta percha, balata and/or polyisoprene. The dispersion compound may comprise any number compositions having suitable mechanical, chemical, radiopacity, anti-microbial and/or anti-inflammatory characteristics. Dispersion compounds according to the present invention may include, but are not necessarily limited to, titanium (particles or elongate strands), crystalline particles, gold (in any form) and/or any mixture of titanium, crystalline particles, and/or gold.

The constituent components cooperate synergistically, lending their individual favorable characteristics to the resulting thermopolymer composition. The favorable characteristics of the thermopolymer matrix may include a relatively low weight, the ability to flow at elevated temperatures, and the ability to conform to a desired shape upon cooling to body temperature. The favorable characteristics of the dispersion compound may include a low reactivity with the human body (i.e., an anti-inflammatory, non-inflammatory and/or non-irritating effect), radiopacity for improved X-ray visualization, and (with regard to titanium or other comparatively high density substances) a high strength-to-weight ratio. The thermopolymer composition of this invention incorporates and capitalizes on the favorable properties of both the thermopolymer matrix and the dispersion compound.

It is an object of this invention to provide a void-filling material that is injectable and moldable. Thermopolymers such as gutta percha have the ability to flow at injection temperatures, and the ability to set in a desired shape when cooled. The thermopolymer of this invention preferably begins to flow above body temperature. The thermopolymer may be mixed with a dispersion compound (such as titanium particles and/or gold) and optionally any desired fillers, heated above body temperature, then injected into the void. The thermopolymer composition will set upon cooling to body temperature, thereby obtaining its resilient, non-dispersing state, and filling the void.

It is another object of this invention to provide a void-filling material that is compatible with the body. Materials that react strongly with the body are prone to degradation, and may also cause an immune response which, in certain instances, causes inflammation. The present invention accomplishes this by providing a dispersion compound comprising titanium and/or gold, both of which are inert compared with other metals and materials. Titanium and gold are therefore less reactive in the body, and less likely to corrode or degrade into substances that might irritate surrounding tissues.

It is a still further object of this invention to provide a void-filling composition that is durable, long lasting, and which minimizes future complications and the need for additional medical procedures. The resiliency of the thermopolymer provides this superior durability, and even more so when augmented with titanium as a dispersion compound.

It is another object of this invention that the void-filling composition is lightweight. Thermopolymers such as gutta percha may constitute a large volume fraction of this composition, and are relatively lightweight. As such, with the relative volume of titanium and/or gold being relatively low (i.e. preferably 5% to 25% by weight relative to the thermopolymer matrix), the weight contribution of the titanium and/or gold is also relatively small. The resulting composition is lightweight, and is therefore less likely to hinder the mobility of joints, appendages, and other body parts in which it is used.

Yet another object of this invention is to provide the thermopolymer composition in a manner that it easy to store and use. The present invention accomplishes this, according to one embodiment, by housing the thermopolymer composition in a compressible tube. The compressible tube and its contents may be heated above body temperature, such as by using hot water, an oven, or an open flame. A force may then be applied to the wall of the tube to compress the tube and discharge its contents through a nozzle. The compressible tube may thereby assist the application of the composition into the void via a small passage creating the opportunity for a large resulting fill.

Alternatively, the void-filling composition may be housed in a syringe instead of a compressible tube. The syringe and its contents may be heated above body temperature, such as by using hot water, an oven, or an open flame. A plunger within the syringe may then be depressed, discharging its contents through a nozzle. The syringe, like the compressible tube, may thereby assist the application of the composition to the void.

According to another feature of the present invention, the titanium particles may include (but are not necessarily limited to) elongate whiskers and/or structurally advantageous reinforcement configurations such as a triangular shape or profile. Providing the titanium as elongate whiskers or such a triangular configuration may further enhance the physical properties of the void-filling composition, taking advantage of various principles of composite material technology.

These and further objects, features, and advantages of the present invention will become apparent from the following detailed description, wherein reference is made to the accompanying figures and drawings.

### BRIEF DESCRIPTION OF THE DRAWINGS

FIG. 1 illustrates a void-filling composition according to a first broad aspect of the present invention;

FIG. 2 illustrates a void-filling composition according to a second broad aspect of the present invention;

FIG. 3 illustrates a compressible tube for storing and delivering a void-filling composition according to another aspect of the present invention; and

FIG. 4 illustrates a syringe for storing and delivering a void-filling composition according to a still further aspect of the present invention.

### DESCRIPTION OF THE SPECIFIC EMBODIMENTS

Illustrative embodiments of the invention are described below. In the interest of clarity, not all features of an actual implementation are described in this specification. It will of course be appreciated that in the development of any such actual embodiment, numerous implementation-specific decisions must be made to achieve the developers' specific goals, such as compliance with system-related and business-related constraints, which will vary from one implementation to another. Moreover, it will be appreciated that such a development effort might be complex and time-consuming, but would nevertheless be a routine undertaking for those of ordinary skill in the art having the benefit of this disclosure. The thermopolymer composition, delivery systems, and related methods disclosed herein boast a variety of inventive features and components that warrant patent protection, both individually and in combination.

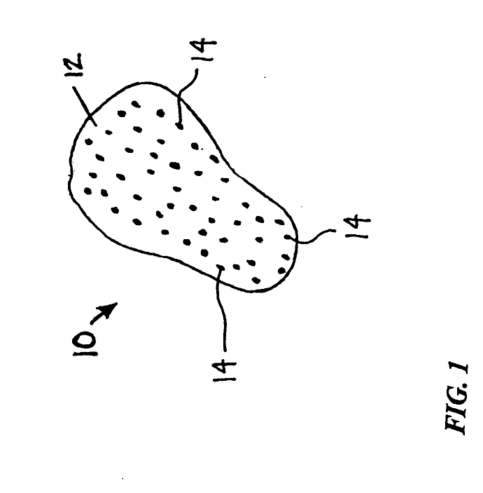

FIG. 1 illustrates a thermopolymer composition 10 according to a first broad aspect of the present invention. The thermopolymer composition 10 is suitable for filling any number of voids (which, as used herein, is defined as any space or gap existing between and/or within biologic structures within a human body). These voids may be formed via natural causes, injury, and/or medical procedures and may, by way of example only, include spaces or gaps formed, created and/or otherwise existing within part or a portion of orthopedic joints (i.e. the discs of the spine and joints of the extremities), bones, muscle and/or viscera. Suitable applications for the thermopolymer composition of the present invention include, but are not limited to, disc nucleus replacement (following partial or full discectomy), vertebroplasty, and tissue augmentation procedures. Illustrative examples of tissue augmentation procedures may include any number of restorative and/or reconfiguration procedures, including but not limited to reconstructive facial surgery, breast augmentation, and urinary incontinence treatment (by injecting the thermopolymer composition of the present invention into the urinary sphincter to serve as a bulking agent).

The thermopolymer composition 10 includes a thermopolymer matrix 12 and a dispersion compound 14. The thermopolymer matrix 12 may comprise any number of suitable thermopolymer materials capable of being heated and injected in a flowable or molten state into a body (either into an existing void or creating a void) and thereafter cooling to body temperature to become a flexible, yet relatively solid material. Because the matrix 12 is a thermoplastic polymer, when cooled to body temperature it returns to its solid state with original solid-state mechanical properties.

The thermopolymer matrix 12 preferably comprises gutta percha, but may also comprise balata, polyisoprene and/or any mixture of gutta percha, balata and/or polyisoprene. Gutta percha is natural latex obtained from certain evergreen trees of East Asia, and has been used in products such as golf-ball coverings, surgical appliances, toys, and adhesives. Balata is a natural rubber obtained from South American trees. Balata, which is sometimes called gutta balata, has propertied similar to those of gutta-percha, and its processing and uses are essentially the same. Polyisoprene, or natural rubber, is harvested from the hevea tree, and has been used to make products such as waterproof boots. Polyisoprene can be treated to give it cross-links, which makes it an even better elastomer.

The dispersion compound 14 may comprise any number compositions having suitable mechanical, chemical, radiopacity, anti-microbial and/or anti-inflammatory characteristics. Dispersion compounds 14 according to the present invention may include, but are not necessarily limited to, titanium (particles or elongate strands), crystalline particles, gold (in any form) and/or any mixture of titanium, crystalline particles, and/or gold. When provided as gold, the dispersion compound 14 may comprise any number of suitable gold-containing compositions, including but not limited to gold particles, strands, and/or gold compositions used for so-called “gold injections” for the treatment of arthritis. The gold composition forming the dispersion compound 14 may constitute between 1 and 40 percent (and more preferably between 3 and 15 percent) by weight of the thermopolymer composition 10.

When provided as titanium particles, the dispersion compound 14 may consist of commercially pure titanium or a titanium alloy with comparable or greater mechanical properties. The titanium particles 14 may constitute between 1 and 50 percent by weight of the thermopolymer composition 10. A titanium-based alloy comprising at least 50 percent by weight titanium included within the scope of “titanium” as used herein. The titanium particles 14 may be substantially spherical, with a diameter less than 50 microns. Preferably, the diameter of the titanium particles is less than 20 microns. The “size” of the titanium particles is defined as the approximate or nominal diameter of the particles. A particle size may be chosen small enough that the resulting composition 10 may be a molecular mixture, with favorable properties and shapes inherent thereto, such as superior mixability with the thermopolymer matrix 12 to facilitate physical properties to meet desired strength characteristics.

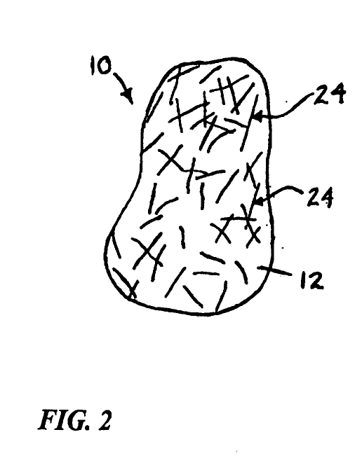

As shown in FIG. 2, the dispersion compound 14 may also comprise elongate titanium whiskers 24 and/or nano and molecularly formed structures (not shown) added to the thermopolymer matrix 12. The titanium whiskers 24 may change the way the composition 10 behaves in its solid state, such as by increasing the modulus of elasticity or tensile strength of the thermopolymer composition 10. The diameter of the titanium whiskers 24 may be between 1 and 50 microns, and the whisker nominal diameter defines the “size” of the titanium whisker particles. The length of the titanium whiskers 24 may be varied to further control the mechanical properties of the composition 10. For example, if the titanium whiskers 24 are long enough to overlap and entangle, the strength of the composition 10 may be greater than if the titanium whiskers 24 are relatively short and distantly spaced. As with the titanium particles or gold described above with reference to FIG. 1, the weight percentage of the titanium whiskers 24 and any additives in composition 10 may be adjusted to optimize the mechanical properties of the composition 10. The optimum length of the titanium whiskers 24 may depend on many factors, including their weight percentage, the part of the body in which the composition 10 will be used, the type of void (i.e. pre-existing, created by an accident or surgery and/or the introduction of the thermopolymer composition 10), as well as the size of the void to be filled.

The thermopolymer composition 10 may also include one or more additives, such as fillers (to reduce the amount of other potential more costly materials), supplemental x-ray contrast agents (to make the composition 10 visible by traditional X-ray), medicinal or pharmaceutical substances (such as antibiotics, anesthetics, and/or biologically transitional material to facilitate biocompatibility), waxes and resins (to increase the flow ability of the composition 10), and sealers (to improve the water-resistance of the composition 10). Zinc may also be added, either to the dispersion compound 14, or separately as additional filler particles, and may comprise up to 10 percent by weight of the composition. Additives should be carefully chosen so the composition 10 retains its beneficial properties such as strength, durability, longevity, and compatibility with the body.

The weight percentage of the dispersion compound 14, thermopolymer matrix 12, and any additives should be chosen to optimize the overall properties of the composition. For example, by increasing the percentage of dispersion compound 14, the strength of the composition 10 may likewise increase, but the weight may also increase, and the flexibility of the composition 10 may decrease. The optimum mix may be determined prior to use of the composition 10, and chosen with respect to a number of factors, including but not limited to the part of the body in which the composition 10 will be used, the type of void (i.e. pre-existing, created by an accident or surgery and/or the introduction of the thermopolymer composition 10), as well as the size of the void to be filled.

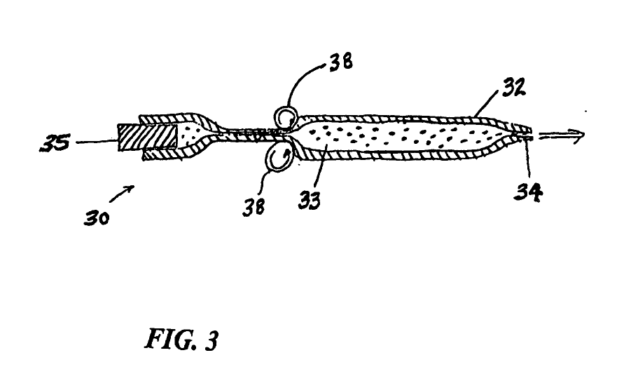

In one embodiment, the thermopolymer composition 10 may be stored in a compressible tube 30, as shown in FIG. 3. The composition 10 may be heated to its fluid state, then poured or otherwise transferred into the compressible tube 30 via the open port, which is subsequently plugged. The end plug 35 may then be installed into the compressible tube 30, and the composition may be allowed to cool to its solid state. When needed, the composition 10 may be reheated to its liquid state from within the compressible tube 30, such as by placing in an oven, in hot water, or over an open flame. The composition 10 may be squeezed from the compressible tube 30, through the nozzle 34, by applying a force to the tube wall 32. The force may be applied to the tube wall 32 either by hand or through mechanical means, such as by using a spring-biased roller 38. The compressible tube 30 may also facilitate the filling the void by transporting the composition 10 into the void.

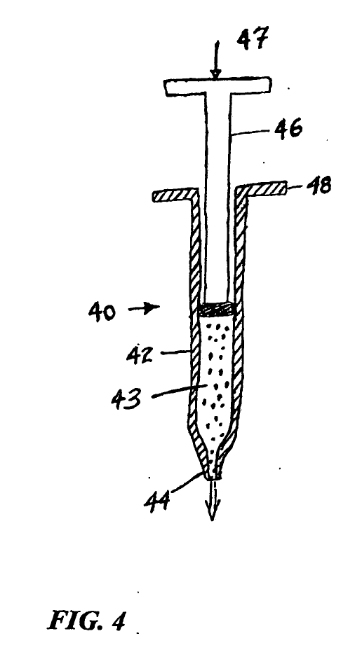

In another embodiment, the composition 10 may instead be stored in a syringe 40, as shown in FIG. 4. The composition 10 may be heated to its fluid state, then poured or otherwise transferred into a body 42 of the syringe 40. The composition 10 may then be allowed to cool to its solid state. When needed, the composition 10 may be reheated to its liquid state from within the syringe 40, such as by placing in an oven, in hot water, or over an open flame. The composition 10 may then be expelled from the syringe 40, through the nozzle 44, by sliding the plunger 46 relative to the body 42 and toward the nozzle 44. A finger stop 48 may be secured to the body 42, such that the body 42 may be held in place while the plunger 47 is depressed. For example, if the syringe 47 is hand-operated, the first and second fingers of one hand may grab the finger stop 48, while the thumb of that hand depresses the plunger 47. The syringe 40 may also facilitate filing the void by transporting the composition 10 into the void.

In a significant aspect of the present invention, the thermoplastic composition 10 may be sterilized before use so as to minimize, if not eliminate, the risk of infecting the patient that may otherwise occur with the introduction of non-sterile compositions during the process of void-filling according to the present invention. Such sterilization techniques may include, but are not necessarily limited to, the application of gamma irradiation to the thermopolymer composition 10 (such as on the order of between 25 to 40 kiloGray). Such irradiation may take place after the formation of the thermopolymer composition 10 and/or after the thermopolymer composition 10 has been introduced into a delivery system such as the compressible tube 30 of FIG. 3 and/or the syringe 40 of FIG. 4.

It may be appreciated that changes to the details of the illustrated embodiments and systems disclosed are possible without departing form the spirit of the invention. While preferred and alternative embodiments of the present invention have been described and illustrated in detail, it is apparent that further modification and adaptations of the preferred and alternative embodiments may occur to those skilled in the art. However, it is to be expressly understood that such modification and adaptations are within the spirit and scope of the present invention, set forth in the following claims.

What is claimed is:

1. A composition for filling a void in a human body, comprising:
a thermopolymer matrix selected from a group consisting of gutta percha, balata, and polyisoprene, and any mixtures thereof; and
a dispersion compound disposed at least partially within said thermopolymer matrix, said dispersion compound comprising at least one of titanium and gold;
wherein the composition has a resilient, non-dispersing state at or below body temperature, and a fluid state after being heated above body temperature, such that the composition may be injected into the void while in the fluid state and thereafter return to the resilient, non-dispersing state.

2. The composition as defined in claim 1, wherein the dispersion compound is less than 50 percent by weight of the composition.

3. The composition as defined in claim 1, wherein the dispersion compound comprises titanium particles forming at least 1 percent by weight of the composition.

4. The composition as defined in claim 3, wherein the dispersion compound comprises titanium particles forming from 20 to 50 percent by weight of the composition.

5. The composition as defined in claim 3, wherein the titanium particles are less than about 20 microns in size.

6. The composition as defined in claim 1, wherein the dispersion compound comprises elongate titanium whiskers.

7. The composition as defined in claim 1, wherein the composition further comprises an additive from a group consisting of a wax and a resin, and any mixtures thereof, to facilitate flow of the composition.

8. The composition as defined in claim 1, wherein the composition further comprises a zinc additive up to 10 percent by weight of the composition.

9. The composition as defined in claim 1, wherein the composition is housed in at least one of a compressible tube and a syringe.

10. The composition as defined in claim 1, wherein the composition is sterilized prior to introduction into the human body.

11. A composition for filling a void in a human body, the composition having a resilient, non-dispersing state at body temperature and fluid state after being heated to a point above body temperature, the composition comprising:
a thermopolymer matrix selected from a group consisting of gutta percha, balata, and polyisoprene, and any mixtures thereof; and
a dispersion compound disposed at least partially within said thermopolymer matrix, said dispersion compound comprising at least one of titanium and gold;
whereby the composition may be heated into said fluid state and introduced into said void while in said fluid state, and thereafter allowed to cool to body temperature and thereby return to said resilient, non-dispersing state to fill said void.

12. The composition as defined in claim 11, wherein the dispersion compound forms from 1 to 50 percent by weight of the composition.

13. The composition as defined in claim 11, wherein the dispersion compound comprises at least one of titanium particles and elongate titanium whiskers.

14. The composition as defined in claim 11, wherein the composition further comprises a zinc additive up to 10 percent by weight of the composition.

15. The composition as defined in claim 11, wherein the composition is sterilized prior to introduction into the human body.

16. A method of preparing a thermopolymer composition, comprising the steps of:
selecting a thermopolymer matrix from a group consisting of gutta percha, balata, and polyisoprene, or any mixture thereof;
selecting a dispersion compound comprising at least one of titanium and gold;
combining the thermopolymer matrix and the dispersion compound to form a thermopolymer composition; and
sterilizing said thermopolymer composition.

17. The method defined in claim 16, wherein the step of sterilizing said thermopolymer composition is accomplished through the use of gamma irradiation.

18. The method defined in claim 17, wherein the gamma irradiation is applied to the thermopolymer composition in the range of between 25 and 40 kiloGray.

19. The method defined in claim 16, further comprising the step of:
including in the composition an additive selected from a group consisting of a wax and a resin, and any mixtures thereof, to facilitate flow of the composition.

20. The method defined in claim 16, wherein the composition is stored in at least one of a compressible tube and a syringe.
